(19) United States
(12) Patent Application Publication
(10) Pub. No.: US 2026/0339390 A1
(43) Pub. Date: Oct. 29, 2026

(54) DETECTION DEVICE AND DETECTION SYSTEM

(71) Applicant: Japan Display Inc., Tokyo (JP)

(72) Inventors: Masahiro TADA, Tokyo (JP); Takenori HIROTA, Tokyo (JP)

(21) Appl. No.: 19/673,367

(22) Filed: May 11, 2026

Related U.S. Application Data
(63) Continuation of application No. PCT/JP2024/037538, filed on Oct. 22, 2024.

(30) Foreign Application Priority Data
Nov. 13, 2023 (JP) 2023-193240

(51) Int. Cl. G06F 1/16; G06F 3/01

(52) U.S. Cl. CPC G06F 1/163; G06F 1/1694; G06F 1/1698; G06F 3/014

(57) ABSTRACT

According to an aspect, a detection device includes: an optical sensor; a detection circuit configured to measure a photocurrent output from the optical sensor; and a storage circuit configured to store pulse wave data of a subject to be detected acquired by the detection circuit. The detection device is configured to calculate a pulse wave interval based on the pulse wave data among pieces of the pulse wave data that corresponds to a predetermined period, store the pulse wave interval in the storage circuit, and output the stored pulse wave interval.

### CROSS-REFERENCE TO RELATED APPLICATION

This application claims the benefit of priority from Japanese Patent Application No. 2023-193240 filed on Nov. 13, 2023 and International Patent Application No. PCT/JP2024/037538 filed on Oct. 22, 2024, the entire contents of which are incorporated herein by reference.

### BACKGROUND

### 1. Technical Field

What is disclosed herein relates to a detection device and a detection system.

### 2. Description of the Related Art

Optical sensors capable of detecting fingerprint patterns and vein patterns are known (for example, Japanese Patent Application Laid-open Publication No. 2009-032005). Such optical sensors are incorporated into wearable devices, such as smartwatches, wristwatches, and wristbands, and used to acquire biometric information such as pulse waves.

According to the disclosures of the following documents: Koichi Fujiwara, "Heart Rate Variability Analysis for Health Monitoring", Systems, control and information, Volume 61, Issue 9; Yoshiaki Matsumoto, Nobuaki Mori, Ryoh Mitajiri, and Zhongwei Jiang, "Study of Mental Stress Evaluation based on analysis of Heart Rate Variability", Life Support, Vol. 22, No. 3, 2010; and Yuka Maeda, Masaki Sekine, Toshiyo Tamura, and Koi-chi Mizutani, "Effect of Pulse Arrival Time Variability on the Difference between Heart Rate Variability and Pulse Rate Variability", Transactions of Japanese Society for Medical and Biological Engineering, 54(6): 261-266, 2016, an autonomic nervous function is evaluated and analyzed using a pulse wave interval (R-R interval (RRI)) acquired based on pulse waves acquired as biometric information.

The acquisition of the pulse wave interval (RRI) as the biometric information is not considered in designing detection devices using such optical sensors.

For the foregoing reasons, there is a need for a detection device and a detection system capable of acquiring the pulse wave interval (RRI) as the biological information.

### SUMMARY

According to an aspect, a detection device includes: an optical sensor; a detection circuit configured to measure a photocurrent output from the optical sensor; and a storage circuit configured to store pulse wave data of a subject to be detected acquired by the detection circuit. The detection device is configured to calculate a pulse wave interval based on the pulse wave data among pieces of the pulse wave data that corresponds to a predetermined period, store the pulse wave interval in the storage circuit, and output the stored pulse wave interval.

According to an aspect, a detection system includes: the detection device; and a server device in a cloud to which the pulse wave interval is transmitted from the detection device via a host. The server device in the cloud is configured to execute calculation of a fast Fourier transform (FFT) using the pulse wave intervals, integrate a predetermined frequency range, and store the result as vital data.

### BRIEF DESCRIPTION OF THE DRAWINGS

FIG. 1 is a schematic view illustrating an exemplary external appearance when a state of a finger accommodated inside a detection device according to an embodiment of the present disclosure is viewed from a lateral side of a housing;

FIG. 2 is a sectional view taken along II-II' in FIG. 1;

FIG. 3 is a sectional view taken along III-III' in FIG. 2;

FIG. 4 is a development view illustrating an exemplary development of a flexible printed circuit board of the detection device according to the embodiment;

FIG. 5 is a schematic top view illustrating an exemplary configuration of the substrate illustrated in FIG. 4;

FIG. 6 is a sectional view taken along VI-VI' in FIG. 5;

FIG. 7 is a block diagram illustrating an exemplary configuration of the detection device according to the embodiment;

FIG. 8 is a circuit diagram illustrating the exemplary configuration of the detection device according to the embodiment;

FIG. 9 is a block diagram illustrating an exemplary configuration of a detection system that includes the detection device according to the embodiment;

FIG. 10 is a flowchart to explain a method for measuring a pulse wave interval (R-R interval (RRI)) and the mean value and the standard deviation of the RRIs of the detection device according to the embodiment;

FIG. 11 is a flowchart to explain a method for frequency analysis of the RRI of the detection device and the detection system according to the embodiment;

FIG. 12 is an explanatory diagram to explain a pulse wave and the RRI measured in the detection device;

FIG. 13 is a graph schematically illustrating a relation between the RRI and time; and

FIG. 14 is an explanatory diagram to explain another example of the method for measuring the RRI and a method for calculating the mean value and the standard deviation of the RRIs.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present disclosure, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### Embodiment

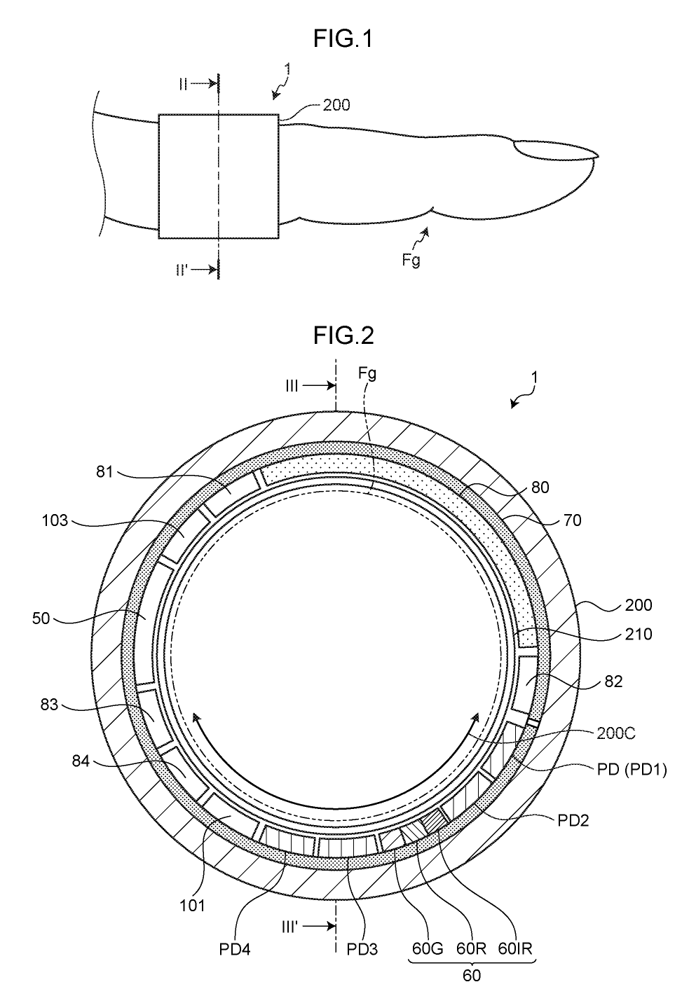

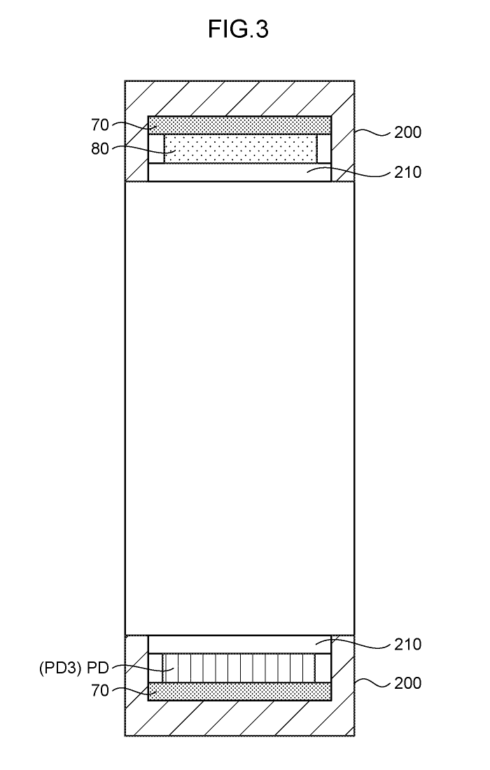

FIG. 1 is a schematic view illustrating an exemplary external appearance when a state of a finger accommodated inside a detection device according to an embodiment of the present disclosure is viewed from a lateral side of a housing. FIG. 2 is a sectional view taken along II-II' in FIG. 1. FIG. 3 is a sectional view taken along III-III' in FIG. 2.

As illustrated in FIG. 1, a detection device 1 according to the embodiment is a finger ring-shaped (ring-shaped) device that can be worn on and removed from a human body. The detection device 1 is worn on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body, and is an object to be measured. The detection device 1 is formed as a finger ring or a wristband so as to be easily carried by a user. In the following description, the detection device 1 is assumed to be used as a finger ring.

The detection device 1 is a ring-shaped device, but is not limited to thereto. The detection device 1 may be built into a wristwatch or a wristband and configured as a wearable device.

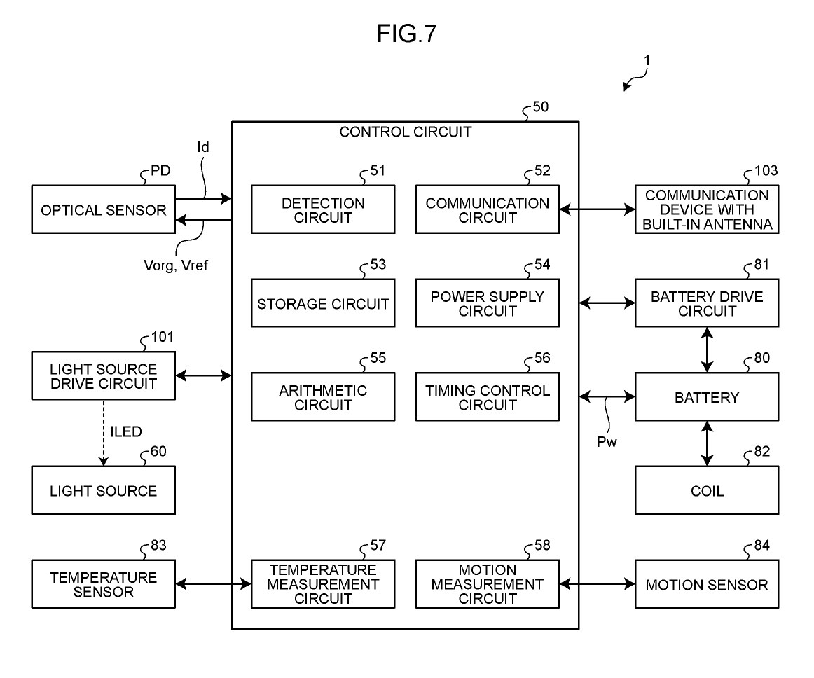

As illustrated in FIG. 2, the detection device 1 includes a housing 200, a protective member 210, a light source 60, a plurality of optical sensors PD1, PD2, PD3, and PD4, and a flexible printed circuit board 70. The detection device 1 further includes a battery 80, a battery drive circuit 81, a coil 82, a light source drive circuit 101, a temperature sensor 83, a motion sensor 84, a communication device 103 with a built-in antenna (hereinafter, refer to as an antenna-integrated communication device 103), and a control circuit 50 that are provided in the housing 200. A detailed configuration example of the battery 80, the battery drive circuit 81, the coil 82, the light source drive circuit 101, the temperature sensor 83, the motion sensor 84, the antenna-integrated communication device 103, and the control circuit 50 will be described later with reference to FIG. 7.

In the following description, the optical sensors PD1, PD2, PD3, and PD4 will each be simply referred to as an optical sensor PD when need not be distinguished from one another.

The housing 200 is a wearable member that is formed in a ring shape (annular shape) wearable on the finger Fg, and is worn on the living body. As illustrated in FIG. 3, the housing 200 has a recessed shape in a sectional view; and the light source 60, the optical sensors PD, the flexible printed circuit board 70, and other components are accommodated in the housing 200. The flexible printed circuit board 70 is formed into a ring shape and accommodated in the housing 200, for example, by forming the housing 200 by filling the area around the ring-shaped flexible printed circuit board 70 with a filling member in a mold. The housing 200 is formed into a ring shape using a member of, for example, a metal or a non-light-transmitting synthetic resin. The housing 200 may, however, be formed of a combination of a plurality of materials, such as a transmissive resin material and a metal material.

The protective member 210 is placed in an opening on an inner peripheral surface side of the housing 200 so as to cover the light source 60, the optical sensors PD, the flexible printed circuit board 70, and other components. The protective member 210 is formed into a ring shape using a housing material of, for example, a light-transmitting synthetic resin or silicon.

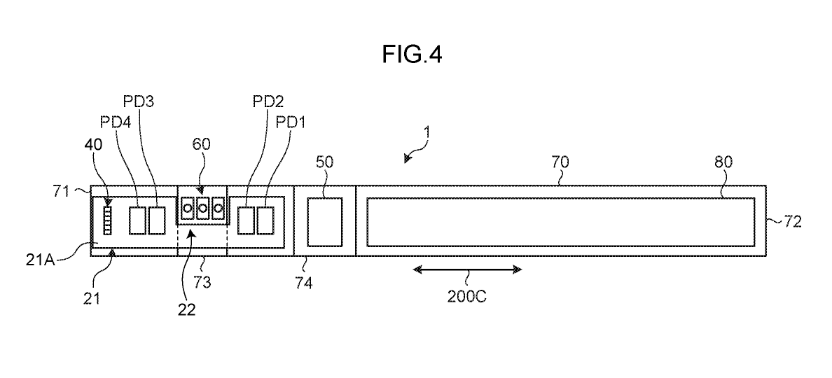

FIG. 4 is a development view illustrating an exemplary development of the flexible printed circuit board of the detection device according to the embodiment. As illustrated in FIG. 4, the flexible printed circuit board 70 is formed in a deformable band shape and is formed into a ring shape by coupling one end 71 to the other end 72. The flexible printed circuit board 70 has a first fabrication area 73 and a second fabrication area 74. The first fabrication area 73 is an area where the light source 60 and other components are fabricated. The second fabrication area 74 is an area where the control circuit 50, the battery 80, and other components are fabricated. Various drive circuits, sensors, and other components fabricated on the flexible printed circuit board 70 illustrated in FIG. 2 are not illustrated in FIG. 4.

A substrate 21 is mounted on the flexible printed circuit board 70 so as to straddle the vicinity of the light source 60 in the first fabrication area 73. The substrate 21 is a sensor board on which the optical sensors PD and other components are fabricated. The substrate 21 is an insulating substrate and is formed, for example, by a film-like resin or the like into a strip-shaped deformable substrate. The flexible printed circuit board 70 electrically couples the light source 60 and the optical sensors PD on the substrate 21 to the control circuit 50.

In the following description, a first direction Dx is one direction in a plane parallel to the substrate 21. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term "plan view" refers to a positional relation as viewed from a direction perpendicular to the substrate 21.

In the present embodiment, the optical sensors PD1 and PD2 and the optical sensors PD3 and PD4 are provided so as to interpose the light source 60 therebetween in a circumferential direction 200C. That is, in the detection device 1, the optical sensors PD1 and PD2, the light source 60, and the optical sensors PD3 and PD4 are arranged in this order in the circumferential direction 200C. Since the optical sensors PD are arranged so as to interpose the light source 60 in the circumferential direction 200C, light emitted by the light source 60 can be detected over a wide area of the housing 200.

The substrate 21 has a notch 22 between the ends of the substrate 21 in the circumferential direction 200C of the housing 200, that is, between the longitudinal ends of the substrate 21. The optical sensors PD3 and PD4 are fabricated on one end 21A side of the substrate 21, and the optical sensors PD1 and PD2 are fabricated on the other end 21B side of the substrate 21 with the notch 22 interposed therebetween. A terminal part 40 is provided at the one end 21A in the longitudinal direction of the substrate 21. The terminal part 40 supplies power from the battery 80 to the optical sensors PD.

In the present embodiment, as illustrated in FIGS. 2 and 3, the flexible printed circuit board 70 is accommodated in the housing 200 such that a front surface provided with the optical sensors PD and the light source 60 are located on an inner peripheral side of the housing 200. If the flexible printed circuit board 70 has a light-transmitting property, the optical sensors PD and the light source 60 may be fabricated on the back surface opposite to the front surface. In this case, the light source 60 only needs to be located so as to emit the light toward the flexible printed circuit board 70 so that the light transmitted through the flexible printed circuit board 70 is emitted toward the outside of the housing 200.

As illustrated in FIG. 2, the light source 60 is provided in the housing 200 and is configured to be capable of emitting the light toward the finger Fg wearing the housing 200. The light source 60 is provided, for example, in an area overlapping the notch 22 of the substrate 21. For example, inorganic light-emitting diodes (LEDs) or organic electroluminescent (EL) diodes (organic light-emitting diodes (OLEDs)) are used as the light source 60. The light source 60 emits light having predetermined wavelengths.

In the present embodiment, the light source 60 includes a light source 60IR, a light source 60R, and a light source 60G. The light source 60IR emits near-infrared light. The light source 60R emits red light. The light source 60G emits green light. The green light has a wavelength of 490 nm to 550 nm, for example. The red light has a wavelength of 640 nm to 770 nm, for example. Infrared light has a wavelength of approximately 2500 nm to approximately 25 μm, for example. The near-infrared light has a wavelength of approximately 770 nm to approximately 2500 nm, for example.

The light sources 60IR, 60R, and 60G are arranged along the circumferential direction 200C of the housing 200. However, the arrangement of the light sources 60IR, 60R, and 60G is merely an example and may be different therefrom. In the following description, the light sources 60IR, 60R, and 60G are each simply referred to as "light source 60" when need not be distinguished from one another. The light source 60 includes the three light sources 60IR, 60R, and 60G, but may include four or more light sources.

The light emitted from the light source 60 is reflected on a surface of a subject to be detected, such as the finger Fg, and enter the optical sensors PD. As a result, the detection device 1 can detect a fingerprint by detecting a shape of asperities on the surface of the finger Fg or the like. The light emitted from the light source 60 may alternatively be reflected in the finger Fg or the like, or transmitted through the finger or the like, and enter the optical sensors PD. Thus, the detection device 1 can detect information on a living body in the finger Fg or the like. Examples of the information on the living body include, but are not limited to, pulse waves, pulsation, and a vascular image of the finger or a palm. That is, the detection device 1 may be configured as a fingerprint detection device to detect the fingerprint or a vein detection device to detect a vascular pattern of, for example, veins.

The detection device 1 of the present embodiment can detect an oxygen saturation level in blood (hereinafter referred to as a "blood oxygen saturation level (SpO₂)") in addition to the pulse waves, the pulsation, and the vascular image as the information on the living body based on the light emitted from the light source 60. Thus, the detection device 1 includes the light sources 60IR, 60R, and 60G as the light source 60 and performs the detection based on the light rays having different wavelengths emitted from these light sources, and thereby can detect the various type of information on the living body. The emission colors of the light sources 60IR, 60R, and 60G described above are examples, and the present disclosure is not limited by the emission colors of the light sources 60IR, 60R, and 60G.

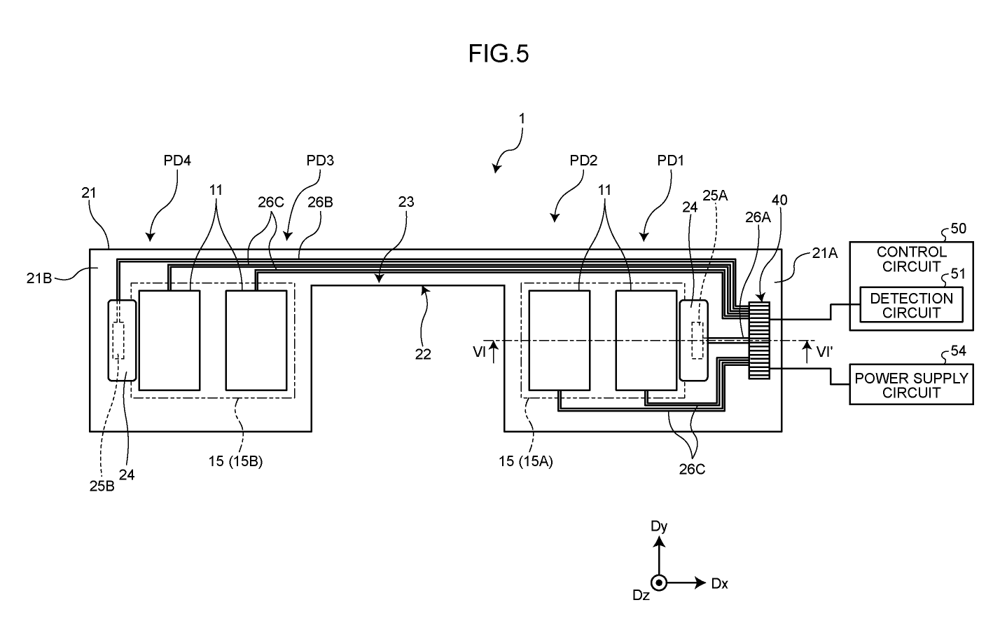

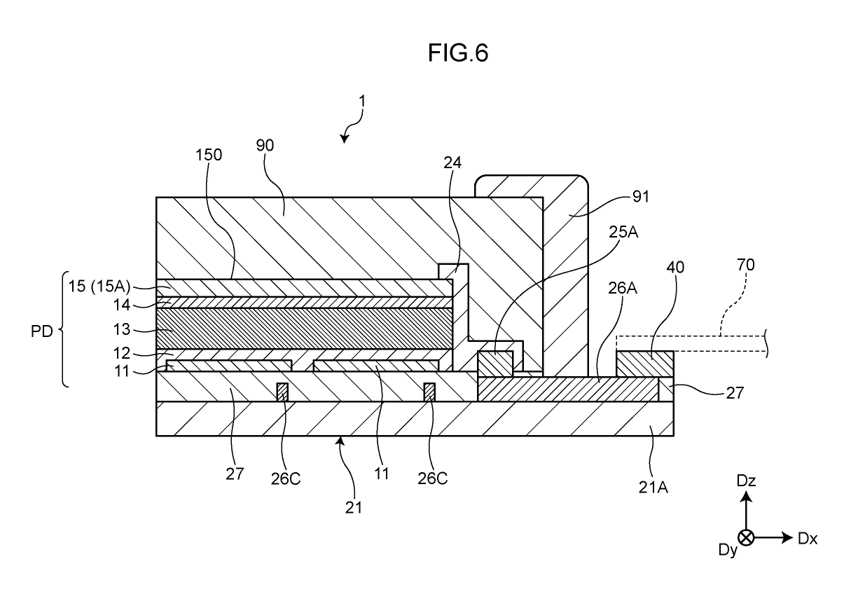

FIG. 5 is a schematic top view illustrating an exemplary configuration of the substrate illustrated in FIG. 4. FIG. 6 is a sectional view taken along VI-VI' in FIG. 5. The detection device 1 includes the optical sensors PD as optical sensor elements. Each of the optical sensors PD outputs an electrical signal corresponding to the light emitted thereto. More specifically, the optical sensor PD is an organic photodiode (OPD).

The optical sensors PD1 and PD2 are provided in the housing 200 so as to be adjacent to one end of the light source 60 in the circumferential direction 200C of the housing 200. The optical sensors PD3 and PD4 are provided in the housing 200 so as to be adjacent to the other end of the light source 60 in the circumferential direction 200C of the housing 200.

As illustrated in FIG. 5, the optical sensors PD1 and PD2 have a configuration in which one upper electrode 15A is stacked on two lower electrodes 11 arranged in the first direction Dx so as to cover the lower electrodes 11. The optical sensors PD3 and PD4 have a configuration in which one upper electrode 15B is stacked on two lower electrodes 11 arranged in the first direction Dx so as to cover the lower electrodes 11. Upper electrodes 15 include the upper electrode 15A and the upper electrode 15B. Each of the upper electrodes 15A and 15B covers the two lower electrodes 11 in plan view. The upper electrodes 15A and 15B are each formed to have a rectangular surface shape and are independent electrodes that are not electrically coupled to each other.

The substrate 21 includes a first power supply electrode 25A and a second power supply electrode 25B that extend along the second direction Dy. The first power supply electrode 25A is provided between the one end 21A side in the first direction Dx of the substrate 21 and the optical sensors PD1 and PD2. The second power supply electrode 25B is provided between the other end 21B side in the first direction Dx of the substrate 21 and the optical sensors PD3 and PD4. The first power supply electrode 25A is electrically coupled to the terminal part 40 of the substrate 21 via a first wiring line 26A and is supplied with a power supply signal from a power supply circuit 54 (refer to FIG. 7) via the terminal part 40. The second power supply electrode 25B is electrically coupled to the terminal part 40 of the substrate 21 via a second wiring line 26B and is supplied with the power supply signal from the power supply circuit 54 via the terminal part 40.

The upper electrode 15A of the optical sensors PD1 and PD2 is coupled to the first power supply electrode 25A via a conductor 24 and electrically coupled to the terminal part 40 via the first wiring line 26A coupled to the first power supply electrode 25A. The upper electrode 15B of the optical sensors PD3 and PD4 is coupled to the second power supply electrode 25B via a conductor 24 and electrically coupled to the terminal part 40 via the second wiring line 26B coupled to the second power supply electrode 25B. With this configuration, the upper electrodes 15A and 15B are supplied with power from independent power systems of the first and the second power supply electrodes 25A and 25B, respectively. Each of the conductors 24 is formed of a conductive material and covers the entire surface of the first power supply electrode 25A or the second power supply electrode 25B, thus electrically coupling the first power supply electrode 25A to the upper electrode 15A or the second power electrode 25B to the upper electrode 15B. The upper electrode 15A and the upper electrode 15B may be directly coupled to the first power supply electrode 25A and the second power supply electrode 25B without the conductors 24 interposed therebetween.

Each of the lower electrodes 11 of the optical sensors PD is coupled to the terminal part 40 via third wiring lines 26C. The third wiring lines 26C of the substrate 21 are coupled to a detection circuit 51 included in the control circuit 50 via the terminal part 40 and signal lines of the flexible printed circuit board 70. In other words, the detection circuit 51 is electrically coupled to the lower electrodes 11 of the optical sensors PD via the signal lines. The detection circuit 51 may be formed as a separate circuit from the control circuit 50.

The first and the second power supply electrodes 25A and 25B are supplied with the power supply signal from the power supply circuit 54 via the terminal part 40, and supply the power supply signal to the upper electrodes 15A and 15B. In the example illustrated in FIG. 5, the first and the second power supply electrodes 25A and 25B are formed in a substantially rectangular shape extending in the second direction Dy in plan view and have the same area (size).

As illustrated in FIG. 5, the substrate 21 has an area of the optical sensors PD1 and PD2 and an area of the optical sensors PD3 and PD4, and is integrally formed into one common substrate. The substrate 21 has the notch 22 formed between the area of the optical sensors PD1 and PD2 and the area of the optical sensors PD3 and PD4 in the first direction Dx. The substrate 21 includes a joint 23 that is in contact with the notch 22 and located between each of the optical sensors PD1 and PD2 and each of the optical sensors PD3 and PD4.

The notch 22 is formed to have a longer length than the length of the light source 60 in the first direction Dx. The notch 22 is formed to have a length longer than the length of the light source 60 and shorter than the length (width) of the substrate 21 in the second direction Dy. The substrate 21 is integrally formed by connecting the area of the optical sensors PD1 and PD2 to the area of the optical sensors PD3 and PD4 via the joint 23 beside the notch 22. The notch 22 is formed in a shape that allows the light source 60 to be located therein. In the present embodiment, the notch 22 is formed into a substantially rectangular shape in plan view, but may have a semicircular, triangular, polygonal, or other shapes, for example. The joint 23 is provided with the second wiring line 26B and the third wiring lines 26C.

As illustrated in FIG. 6, the third wiring lines 26C are provided on the upper surface of the substrate 21. The third wiring lines 26C are formed, for example, of a metal, and is formed of a material having better conductivity than the lower electrodes 11 of the optical sensor PD. The third wiring lines 26C are provided in a layer between the substrate 21 and the optical sensor PD in the third direction Dz. The third wiring lines 26C are electrically coupled to the terminal part 40 on the substrate 21 (refer to FIG. 5). The third wiring lines 26C may be formed, for example, in the same layer as the lower electrodes 11, and may be formed of a metal. An insulating layer 27 is provided on the substrate 21 so as to cover the third wiring lines 26C. The insulating layer 27 may be an inorganic insulating film or an organic insulating film.

The optical sensor PD is provided as a sensor element on the insulating layer 27. The optical sensor PD includes the lower electrodes 11, a lower buffer layer 12, an active layer 13, an upper buffer layer 14, and the upper electrode 15 (upper electrode 15A). In the optical sensor PD, the lower electrodes 11, the lower buffer layer 12, the active layer 13, the upper buffer layer 14, and the upper electrode 15 are stacked in this order in the third direction Dz perpendicular to the substrate 21.

The lower electrode 11 is formed, for example, of a light-transmitting conductive material such as indium tin oxide (ITO).

The active layer 13 changes in characteristics (for example, voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 and the upper buffer layer 14 are provided to facilitate holes and electrons generated in the active layer 13 to reach the lower electrode 11 or the upper electrode 15. One of the lower buffer layer 12 and the upper buffer layer 14 is a hole transport layer. The other of the lower buffer layer 12 and the upper buffer layer 14 is an electron transport layer. The material of the hole transport layer is a metal oxide layer. For example, tungsten oxide (WO₃) or molybdenum oxide is used as the metal oxide layer. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is continuously formed over the entire optical sensors PD. In other words, the upper electrode 15 is continuously provided on the optical sensors PD. The upper electrode 15 faces the lower electrodes 11 with the lower buffer layer 12, the active layer 13, and the upper buffer layer 14 interposed therebetween. The upper electrode 15 is formed, for example, of a light-transmitting conductive material such as ITO or indium zinc oxide (IZO). A portion of an end of an upper surface 150 of the upper electrode 15 is electrically coupled to the conductor 24. The conductor 24 is electrically coupled to the first power supply electrode 25A and supplies the power signal from the first power supply electrode 25A to the upper electrode 15.

A sealing film 90 is provided on the upper electrode 15. An inorganic film, such as a silicon nitride film or an aluminum oxide film, or a resin film, such as an acrylic film, is used as the sealing film 90. The sealing film 90 is not limited to a single layer, but may be a multilayered film having two or more layers obtained by combining the inorganic film with the resin film mentioned above. The sealing film 90 well seals the optical sensor PD, and thus can inhibit moisture from entering the optical sensor PD from the upper surface side thereof. In the present embodiment, the optical sensor PD is configured to protect the terminal part 40, the substrate 21, and other components by covering from the sealing film 90 to part of the terminal part 40 with resin 91.

FIG. 7 is a block diagram illustrating an exemplary configuration of the detection device according to the embodiment. As illustrated in FIG. 7, the control circuit 50 outputs a control signal to each of the optical sensors PD, the battery 80, the battery drive circuit 81, the coil 82, the light source drive circuit 101, the temperature sensor 83, the motion sensor 84, and the antenna-integrated communication device 103 to control operations of those various components. The control circuit 50 includes, for example, the detection circuit 51, a communication circuit 52, a storage circuit 53, the power supply circuit 54, an arithmetic circuit 55, a timing control circuit 56, a temperature measurement circuit 57, and a motion measurement circuit 58.

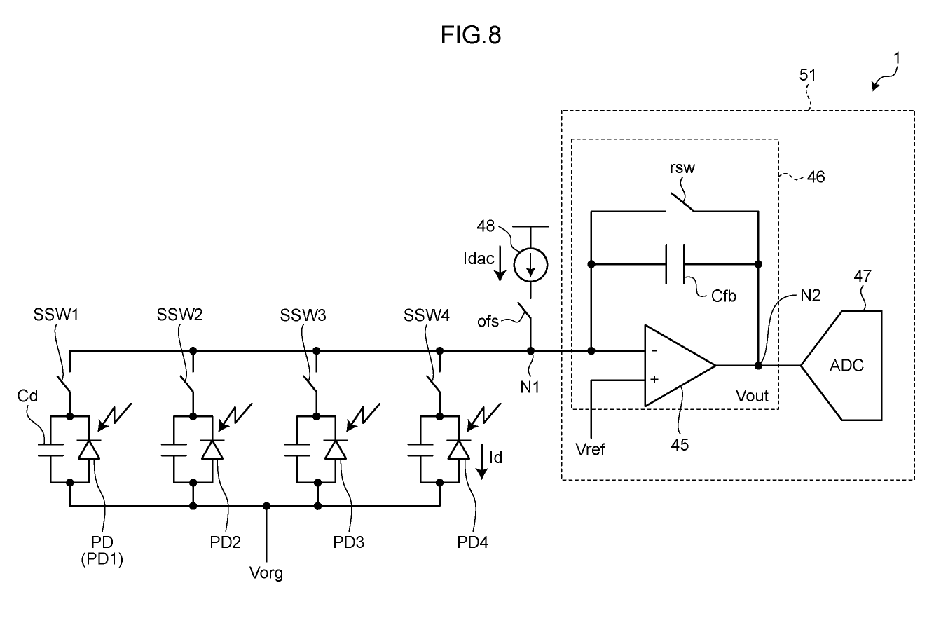

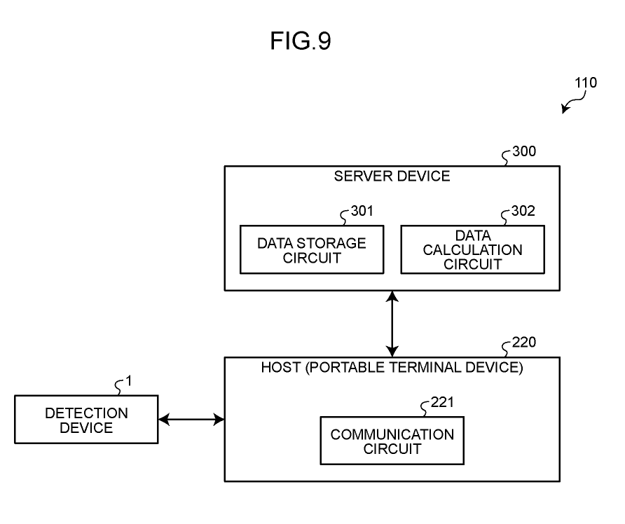

The detection circuit 51 is a current detection circuit that measures a photocurrent Id output from the optical sensor PD. The detection circuit 51 is configured, for example, with an integrating circuit 46 and an analog-to-digital (A/D) conversion circuit 47 (refer to FIG. 8). The detection circuit 51 measures the photocurrent Id output from the optical sensor PD, performs signal processing such as A/D conversion, and outputs a sensor value (pulse wave) corresponding to the photocurrent Id to a host 220 (refer to FIG. 9).

The light source drive circuit 101 outputs a light source drive signal ILED to the light source 60 based on the control signal from the control circuit 50, and controls on and off of the light source 60.

The temperature sensor 83 and the temperature measurement circuit 57 measure the temperature (body temperature) of the subject to be detected. The motion sensor 84 and the motion measurement circuit 58 detect the posture and the motion of the subject to be detected. The motion sensor 84 may have any configuration depending on the mode of use of the detection device 1 and the required detection accuracy, and is configured, for example, with a three-axis acceleration sensor, a three-axis gyro sensor, and a three-axis geomagnetic sensor.

The communication circuit 52 and the antenna-integrated communication device 103 wirelessly transmit and receive various types of information to and from the external host 220 (refer to FIG. 9). The communication circuit 52 and the antenna-integrated communication device 103 support wireless communication standards. Examples of the communication standards include, but are not limited to, Near-field communication (NFC) standards and cellular phone communication standards, such as 3G, 4G, and 5G. The communication circuit 52 and the antenna-integrated communication device 103 provide the received information to various components of the control circuit 50. The communication circuit 52 and the antenna-integrated communication device 103 transmit the various types of information acquired by the optical sensor PD, the temperature sensor 83, and the motion sensor 84 to the external host 220 (refer to FIG. 9).

The battery drive circuit 81 is a circuit that controls charging of the battery 80. The coil 82 is a coil for charging the battery 80. The battery 80 of the detection device 1 is configured to be chargeable by the coil 82 in a contactless manner. The power supply circuit 54 of the control circuit 50 supplies reference potentials Vorg and Vref to the optical sensor PD and supplies the light source drive signal ILED to the light source 60, based on power Pw from the battery 80. The power supply circuit 54 supplies power to the communication circuit 52, the temperature measurement circuit 57, the motion measurement circuit 58, and other components, as needed.

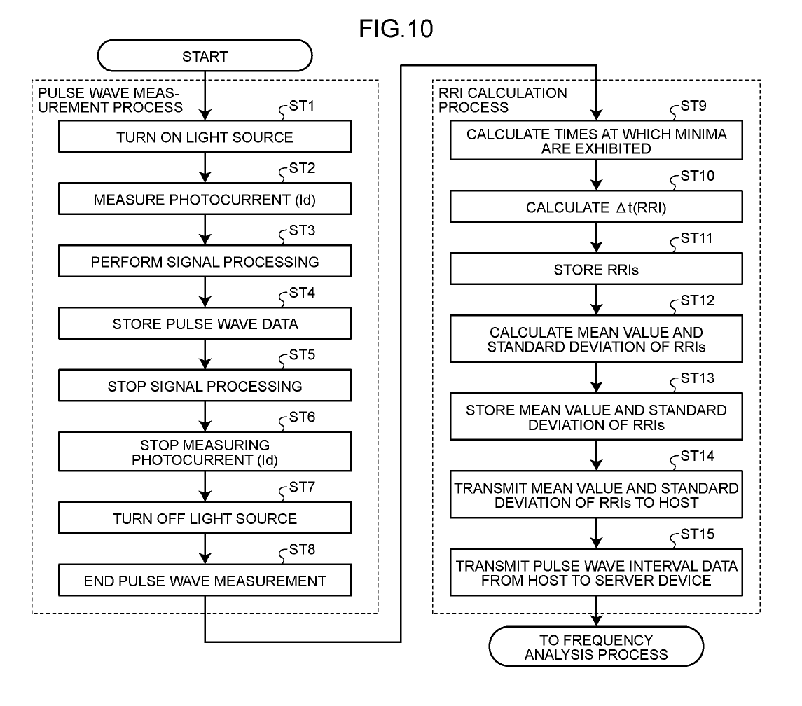

The storage circuit 53 of the control circuit 50 temporarily stores therein the information, such as the pulse waves and other information, measured by the detection circuit 51. The arithmetic circuit 55 calculates a pulse wave interval (hereinafter, referred to as "R-R interval (RRI)") and the mean value, the standard deviation, and other information of the RRI based on the information on the pulse waves and other information measured by the detection circuit 51. The pulse wave interval (RRI), and the mean value and the standard deviation of the RRIs will be described later with reference to FIG. 10 and subsequent drawings.

The timing control circuit 56 of the control circuit 50 controls the various components of the control circuit 50 to operate synchronously or non-synchronously.

FIG. 8 is a circuit diagram illustrating the exemplary configuration of the detection device according to the embodiment. As illustrated in FIG. 8, the optical sensors PD1, PD2, PD3, and PD4 are coupled to the integrating circuit 46 included in the detection circuit 51 via coupling switches SSW1, SSW2, SSW3, and SSW4. The detection device 1 includes a constant current source 48. The constant current source 48 is coupled to a coupling node N1 between the optical sensors PD1, PD2, PD3, PD4 and the integrating circuit 46 via an offset switch ofs.

In the following description, the coupling switches SSW1, SSW2, SSW3, and SSW4 will each be simply referred to as a "coupling switch SSW" when need not be distinguished from one another.

The anode of the optical sensor PD is supplied with the reference potential Vorg from the power supply circuit 54. The cathode of the optical sensor PD is coupled to the detection circuit 51 via the coupling switch SSW.

Sensor capacitance Cd is coupled in parallel to the optical sensor PD. The sensor capacitance Cd is capacitance generated between the upper electrode 15 and the lower electrode 11 of the optical sensor PD.

The detection circuit 51 includes the integrating circuit 46 and the A/D conversion circuit 47. The integrating circuit 46 converts the variation of the photocurrent Id output from the optical sensor PD into a voltage variation. The A/D conversion circuit 47 converts analog signals output from the integrating circuit 46 into digital signals.

The integrating circuit 46 includes an operational amplifier 45, a feedback capacitor Cfb, and a reset switch rsw. The inverting input (-) of the operational amplifier 45 is coupled to the optical sensor PD via the coupling switch SSW. The optical sensors PD1, PD2, PD3, and PD4 are coupled in parallel to the inverting input (-) of the integrating circuit 46 via the coupling switches SSW1, SSW2, SSW3, and SSW4.

The non-inverting input (+) of the operational amplifier 45 is supplied with the reference potential Vref from the power supply circuit 54. The output of the operational amplifier 45 is coupled to the A/D conversion circuit 47. An output voltage Vout of the integrating circuit 46 is a voltage value at a coupling node N2 between the operational amplifier 45 and the A/D conversion circuit 47.

In the present embodiment, when the optical sensor PD is irradiated with the light in an exposure period, a current corresponding to the light intensity flows through the optical sensor PD. As a result, an electric charge is stored in the sensor capacitance Cd.

During a predetermined period including the exposure period, the reset switch rsw is turned on and the integrating circuit 46 is reset. Before a readout period, the reset switch rsw is turned off and the integrating circuit 46 is released from the reset state.

Turning on the coupling switch SSW starts the readout period, and a current corresponding to the electric charge stored in the sensor capacitance Cd flows to the integrating circuit 46 of the detection circuit 51. As a result, the photocurrent Id is calculated. With an offset current Idac, the current flowing in the integrating circuit 46 is adjusted.

The A/D conversion circuit 47 performs signal processing on the output voltage Vout output from the integrating circuit 46 and outputs the sensor value corresponding to the photocurrent Id to the host 220.

FIG. 9 is a block diagram illustrating an exemplary configuration of a detection system that includes the detection device according to the embodiment. As illustrated in FIG. 9, a detection system 110 including the detection device 1 includes the host 220 and a server device 300 in a cloud that is provided so as to be connectable to the host 220. The host 220 is, for example, a portable terminal device, such as a smartphone, a cellular phone, or a tablet terminal device. A communication circuit 221 of the host 220 is capable of wirelessly communicating with the detection device 1 and the server device 300. The server device 300 is not limited to being provided in the cloud.

The server device 300 includes, for example, a data storage circuit 301 and a data calculation circuit 302. The information on the subject to be detected measured by the detection device 1, such as the data on the RRI, and the mean value and the standard deviation of the RRIs, is transmitted to the server device 300 via the host 220. The various types of data transmitted to the server device 300 are stored in the data storage circuit 301. The data storage circuit 301 stores therein not only the RRI, but also the various types of biometric information on the subject to be detected as vital data. The data calculation circuit 302 performs calculation processing on the RRI, and the calculation results are stored in the data storage circuit 301.

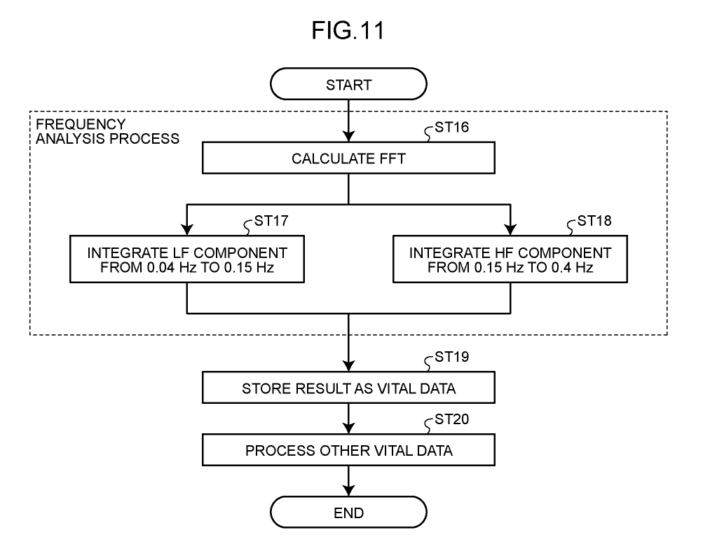

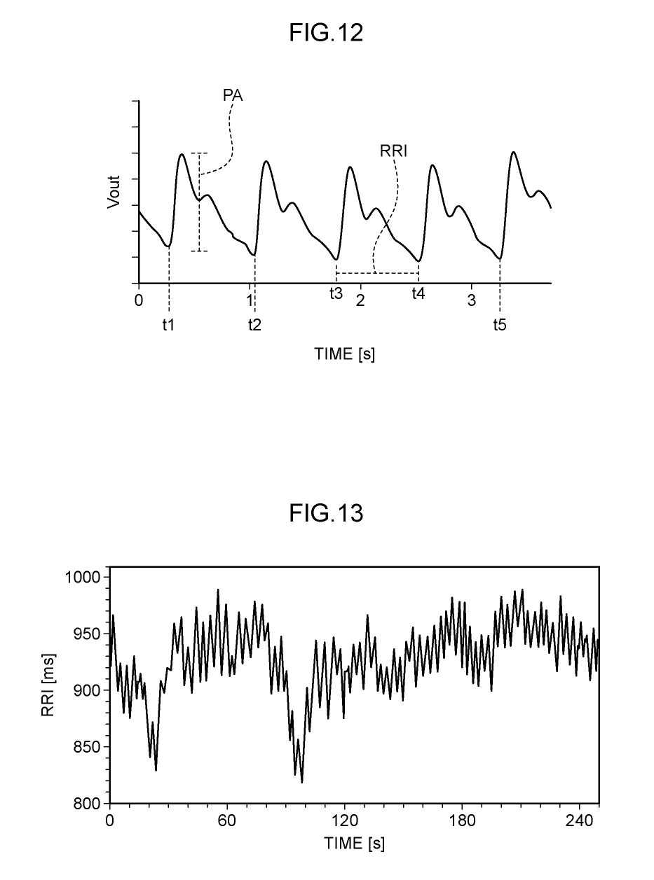

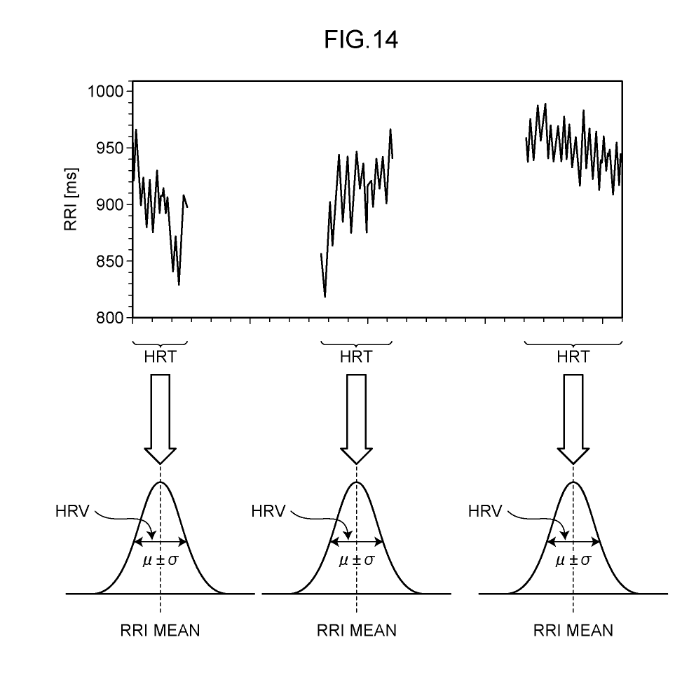

The following describes a method for measuring the pulse wave interval (RRI) and a method for acquiring the various types of data using the detection device 1 and the detection system 110. FIG. 10 is a flowchart to explain the method for measuring the RRI and the mean value and the standard deviation of the RRIs of the detection device according to the embodiment. FIG. 11 is a flowchart to explain a method for frequency analysis of the RRI of the detection device and the detection system according to the embodiment. FIG. 12 is an explanatory diagram to explain the pulse wave and the RRI measured in the detection device. FIG. 13 is a graph schematically illustrating a relation between the RRI and time. FIG. 14 is an explanatory diagram to explain another example of the method for measuring the RRI and a method for calculating the mean value and the standard deviation of the RRIs.

As illustrated in FIG. 10, the detection device 1 drives the optical sensor PD and the light source 60 to measure the pulse wave data of the subject to be detected (pulse wave measurement process). First, the light source drive circuit 101 turns on the light source 60 based on the control signal from the control circuit 50 (Step ST1). The light source drive circuit 101 turns on, for example, the light source 60G, and the light source 60G emits the green light.

The photocurrent Id flows through the optical sensor PD, which corresponds to the light emitted from the light source 60. The detection circuit 51 measures the photocurrent Id output from the optical sensor PD (Step ST2). The detection circuit 51 performs the signal processing described above on the measured photocurrent Id (Step ST3).

In the graph illustrated in FIG. 12, the vertical axis is the output voltage Vout of the detection circuit 51 (integrating circuit 46) and the horizontal axis is time (s). As illustrated in FIG. 12, the detection circuit 51 measures the pulse wave data of the subject to be detected based on the photocurrent Id from the optical sensor PD. The pulse wave data has a waveform in which maxima and minima of the output voltage Vout are cyclically repeated. The storage circuit 53 (refer to FIG. 7) stores therein the pulse wave data acquired by the detection circuit 51 (Step ST4).

Then, the coupling switch SSW (refer to FIG. 8) turns off to end the readout period, and the detection circuit 51 stops the signal processing on the photocurrent Id (Step ST5), and also stops measuring the photocurrent Id from the optical sensor PD (Step ST6). The light source drive circuit 101 turns off the light source 60 based on the control signal from the control circuit 50 (Step ST7).

As described above, the detection device 1 stops driving the optical sensor PD and the light source 60, and ends the measurement of the pulse wave data of the subject to be detected (Step ST8).

Then, the arithmetic circuit 55 (refer to FIG. 7) calculates the RRI and the various types of data using the pulse wave data acquired at Steps ST1 to ST8 (RRI calculation process).

First, the arithmetic circuit 55 calculates times at which the acquired pulse wave data (refer to FIG. 12) exhibits minima (Step ST9). In the example illustrated in FIG. 12, the arithmetic circuit 55 calculates times t1, t2, t3, t4, t5, ... at which the output voltage Vout exhibits the minima.

The arithmetic circuit 55 calculates a time interval Δt(RRI) between the adjacent minima of the output voltage Vout (Step ST10). In the present disclosure, the pulse wave interval (RRI) is the time interval Δt between the adjacent minima of the output voltage Vout in the pulse wave data. In the example illustrated in FIG. 12, the time interval Δt(RRI) is calculated as each of Δt(RRI) = t2 - t1, t3 - t2, t4 - t3, and t5 - t4. FIG. 12 illustrates Δt(RRI) = t4 - t3 as an example of the RRI period. The arithmetic circuit 55 may calculate other various types of data, such as amplitude PA of the pulse wave, as needed at Step ST10.

As illustrated in FIG. 13, the arithmetic circuit 55 may calculate the RRI for each beat during a continuous predetermined period (period from 0 to 250 seconds). Alternatively, as illustrated in FIG. 14, the arithmetic circuit 55 may calculate the RRI based on the pulse wave data corresponding to a predetermined measurement period HRT. The measurement period HRT is, for example, 30 seconds or longer. That is, the arithmetic circuit 55 has the measurement period HRT during which the RRI is calculated and a stop period (for example, 60 seconds or longer) during which the calculation of the RRI stops, and alternately executes the calculation of the RRI during the measurement period HRT and the predetermined stop interval. In this case, the detection device 1 can reduce power consumption while acquiring the data required to analyze the RRI.

The storage circuit 53 stores therein the RRIs obtained at Steps ST9 and ST10 (Step ST11). The storage circuit 53 stores therein the RRIs in association with the respective measurement times, as illustrated in FIG. 13. Alternatively, the storage circuit 53 stores therein the RRIs in association with the respective the measurement periods HRT, as illustrated in FIG. 14.

The arithmetic circuit 55 calculates the mean value and the standard deviation of the RRIs based on the stored RRIs (Step ST12). FIG. 14 illustrates an example of calculating the mean value (μ) and the standard deviation (σ) of the RRIs for each measurement period HRT. The detection device 1 can obtain, for example, heart rate variability (HRV) based on the mean value (μ) and the standard deviation (σ) of the RRIs. Heart rate variability (HRV) is expressed as μ ± σ, and is used to analyze autonomic nerve activities.

The storage circuit 53 stores therein the mean value (μ) and the standard deviation (σ) of the RRIs obtained at Step ST12 (Step ST13).

The control circuit 50 transmits the RRIs, and the mean value and the standard deviation of the RRIs obtained at Steps ST9 to ST13 to the host 220 (refer to FIG. 9) via the communication circuit 52 (refer to FIG. 7) (Step ST14). In the following description, the various types of data related to the RRIs including the RRIs, and the mean value and the standard deviation of the RRIs may be referred as "pulse wave interval data".

The host 220 transmits, to the server device 300 in the cloud, the pulse wave interval data (the RRIs, and the mean value and the standard deviation of the RRIs) acquired from the detection device 1 (Step ST15).

As illustrated in FIG. 11, the server device 300 in the cloud analyzes the pulse wave interval data acquired at Step ST15 (frequency analysis process). The data calculation circuit 302 included in the server device 300 in the cloud executes calculation of a fast Fourier transform (FFT) using the RRIs acquired at Step ST15 (Step ST16). Thus, the data calculation circuit 302 calculates a frequency variance of the RRIs.

The data calculation circuit 302 of the server device 300 integrates a predetermined frequency range (from 0.04 Hz to 0.15 Hz) based on the frequency variance of the RRIs (Step ST17) and stores the result as the vital data in the data storage circuit 301 (Step ST19). The data calculation circuit 302 of the server device 300 integrates a predetermined frequency range (from 0.15 Hz to 0.4 Hz) based on the frequency variance of the RRIs (Step ST18) and stores the result as the vital data in the data storage circuit 301 (Step ST19). By setting the measurement period HRT of the RRIs illustrated in FIG. 14, for example, to 30 seconds or longer, the integration of the frequency range illustrated at Step ST17 can be performed well.

The server device 300 stores therein the pulse wave interval data (mean value and standard deviation of RRIs) acquired at Step ST15 as the vital data. The server device 300 acquires not only the pulse wave interval data, but also the other vital data, from the detection device 1 or the host 220, and performs data processing on the other vital data using the data calculation circuit 302 (Step ST20). The other vital data is also stored in the data storage circuit 301.

Examples of the other vital data include, but are not limited to, at least one of the following: data of the blood oxygen saturation level (SpO₂) detected by the detection device 1; data acquired by the motion sensor 84; and data acquired by the temperature sensor 83. The other vital data is transmitted from the detection device 1 via the host 220 to the server device 300 in the cloud.

The other vital data may include a sleep duration, the metabolic equivalent of tasks (METs) (physical activity level), the number of steps, a distance traveled, calories, and so forth. The vital data is detected by at least one of the optical sensor PD, the temperature sensor 83, and the motion sensor 84.

As described above, the detection device 1 and the detection system 110 measure the pulse wave data of the subject to be detected using the optical sensor PD, and calculates the pulse wave interval (RRI) based on the pulse wave data among pieces of the pulse wave data that corresponds to the predetermined period. The pulse wave interval data (the RRIs, and the mean value and the standard deviation of the RRIs) is transmitted to the server device 300 in the cloud via the host 220. The detection device 1 and the detection system 110 can monitor the physical and mental condition of the subject to be detected by acquiring the pulse wave interval data (the RRIs, and the mean value and the standard deviation of the RRIs). The detection device 1 and the detection system 110 can also, for example, analyze the autonomic nerve activities and predict various physical and mental disorders using the pulse wave interval data (the RRIs, and the mean value and the standard deviation of the RRIs).

The method for measuring the RRI and the method for acquiring the various types of data that are illustrated in FIGS. 10 -14 are merely exemplary, and can be changed as appropriate. The RRI is defined as the time interval Δt between the adjacent minima in the pulse wave data, but is not limited to this time interval. For example, the RRI may be the time interval Δt between adjacent maxima in the pulse wave data. At Steps ST14 and ST15 illustrated in FIG. 10, an example is illustrated in which the RRIs, and the mean value and the standard deviation of the RRIs are transmitted as the pulse wave interval data, but the present disclosure is not limited to this example. The detection device 1 may transmit at least the RRIs and at least one of the mean value and the standard deviation of the RRIs.

The case has been described where the detection device 1 is configured such that the substrate 21 and other components are accommodated in the ring-shaped housing 200, but the present disclosure is not limited to this case. The detection device 1 may be configured, for example, such that those components are accommodated in a rectangular housing or attached to the subject to be measured without being accommodated in the housing.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiment and the modifications thereof described above.

What is claimed is:

1. A detection device comprising:
an optical sensor;
a detection circuit configured to measure a photocurrent output from the optical sensor; and
a storage circuit configured to store pulse wave data of a subject to be detected acquired by the detection circuit, wherein
the detection device is configured to calculate a pulse wave interval based on the pulse wave data among pieces of the pulse wave data that corresponds to a predetermined period, store the pulse wave interval in the storage circuit, and output the stored pulse wave interval.

2. The detection device according to claim 1, wherein the pulse wave interval is a time interval between adjacent minima in the pulse wave data.

3. The detection device according to claim 1, wherein the predetermined period is 30 seconds or longer.

4. The detection device according to claim 1, configured to alternately execute a period in the predetermined period during which the pulse wave interval is calculated and a stop period during which the calculation of the pulse wave interval stops.

5. The detection device according to claim 1, configured to calculate at least one of a mean value or a standard deviation of the pulse wave intervals in the predetermined period based on the pulse wave intervals, and output the at least one of the mean value or the standard deviation of the pulse wave intervals.

6. A detection system comprising:
the detection device as claimed in claim 1; and
a server device in a cloud to which the pulse wave interval is transmitted from the detection device via a host, wherein
the server device in the cloud is configured to execute calculation of a fast Fourier transform (FFT) using the pulse wave intervals, integrate a predetermined frequency range, and store the result as vital data.

7. The detection system according to claim 6, wherein
the detection device is configured to calculate at least one of a mean value or a standard deviation of the pulse wave intervals in the predetermined period based on the pulse wave intervals, and
the at least one of the mean value or the standard deviation of the pulse wave intervals is transmitted to the server device in the cloud from the detection device via the host.

8. The detection system according to claim 6, wherein the predetermined frequency range is 0.04 Hz to 0.15 Hz.

9. The detection system according to claim 6, wherein the predetermined frequency range is 0.15 Hz to 0.4 Hz.

10. The detection system according to claim 6, wherein the detection device is a ring-shaped device.

11. The detection system according to claim 6, wherein the detection device is built into a wristwatch or a wristband.

12. The detection system according to claim 6, wherein the optical sensor is an organic photodiode (OPD).

13. The detection system according to claim 6, further comprising at least one of a motion sensor or a temperature sensor, wherein
at least one of data acquired by the motion sensor, data acquired by the temperature sensor, or SpO₂ data acquired by the optical sensor is transmitted to the server
device in the cloud.
